(19) United States
(12) Patent Application Publication
(10) Pub. No.: US 2017/0158591 A1
(43) Pub. Date: Jun. 8, 2017

(54) SYNTHESIS OF DIACETONE ALCOHOL AND MESITYL OXIDE

(71) Applicant: SABIC GLOBAL TECHNOLOGIES B.V., BERGEN OP ZOOM (NL)

(72) Inventors: Emmanuel Yaw OSEI-TWUM, RIYADH (SA); Nouri HASSAN, RIYADH (SA); Nagmeddin ELWAER, RIYADH (SA); Ganesh Shivaram BHAT, RIYADH (SA); Carlos Godinez SEOANE, Cartagena (ES); Khalid H. AL-ASSAF, RIYADH (SA)

(21) Appl. No.: 15/325,502

(22) PCT Filed: Jul. 23, 2015
(86) PCT No.: PCT/IB2015/055591

§ 371 Date: Jan. 11, 2017

Related U.S. Application Data
(60) Provisional application No. 62/029,203, filed on Jul. 25, 2014.

(51) Int. Cl. C07C 45/72; C07C 45/74

(52) U.S. Cl. CPC C07C 45/72; C07C 45/74

(57) ABSTRACT

Processes for synthesizing diacetone alcohol from acetone are provided. An exemplary process includes contacting acetone with a heterogeneous catalyst at a temperature of less than 100° C., to provide diacetone alcohol. The heterogeneous catalyst can include molecular sieves. Processes for synthesizing mesityl oxide from acetone are also provided.

### BACKGROUND

This application relates to processes for synthesis of diacetone alcohol and mesityl oxide. Diacetone alcohol, also known as 4-hydroxy-4-methylpentan-2-one and 4-hydroxy-4-methyl-2-pentanone, is a keto-alcohol with the formula CH₃C(O)CH₂C(OH)(CH₃)₂. Diacetone alcohol can be useful as a solvent for various processes and can also serve as a synthetic intermediate for preparation of other compounds. Diacetone alcohol can also be incorporated into various lacquers, wood treatments, coatings, cleaning agents, and other materials.

Mesityl oxide, also known as 4-methylpent-3-en-2-one and 4-methyl-3-penten-2-one, is an α,β-unsaturated ketone with the formula CH₃C(O)CH═C(CH₃)₂. Like diacetone alcohol, mesityl oxide can be useful as a solvent for various processes and can also serve as a synthetic intermediate for preparation of other compounds.

Diacetone alcohol and mesityl oxide can be synthesized from acetone (propanone), for example according to the following general synthetic scheme.

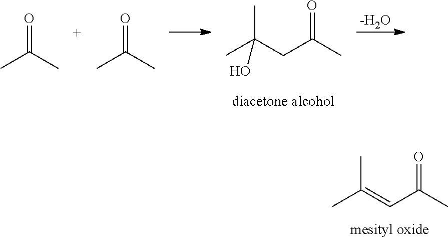

One equivalent of acetone can react with a second equivalent of acetone in an aldol reaction to provide diacetone alcohol. Dehydration of diacetone alcohol can provide mesityl oxide.

Diacetone alcohol and mesityl oxide can be prepared from acetone using base catalysis. Synthesis of diacetone alcohol and mesityl oxide from acetone using heterogeneous catalysts has been described. For example, U.S. Pat. No. 5,292,980 to Dessau, U.S. Pat. No. 8,697,924 to Bauldreay et al., and U.S. Patent Application Publication No. 2013/0185922 A1 to Cortright and Blommel disclose preparation of diacetone alcohol and mesityl oxide from acetone using heterogeneous aluminosilicate catalysts at high temperatures (100° C. or higher). Others have described synthesis of diacetone alcohol and mesityl oxide from acetone using heterogeneous solid base catalysts. See, for example, U.S. Pat. No. 5,672,764 to Teissier et al. and International Patent Application Publication No. WO/2004/101485.

There nonetheless remains a need for improved processes for synthesizing diacetone alcohol and mesityl oxide with one or more of greater yield, greater selectivity, greater efficiency, greater economy, reduced input of energy, and milder conditions.

### SUMMARY

Disclosed herein are processes for synthesizing diacetone alcohol. In some embodiments, an exemplary process for synthesizing diacetone alcohol includes contacting acetone with a neutral heterogeneous catalyst at a temperature of less than 100° C., to provide diacetone alcohol. In some embodiments, an exemplary process for synthesizing diacetone alcohol includes contacting acetone with molecular sieves, to provide diacetone alcohol.

Also disclosed are processes for synthesizing mesityl oxide. In some embodiments, an exemplary process for synthesizing mesityl oxide includes contacting acetone with a neutral heterogeneous catalyst at a temperature of less than 100° C., to provide mesityl oxide. In some embodiments, an exemplary process for synthesizing mesityl oxide includes contacting acetone with molecular sieves, to provide mesityl oxide.

In certain embodiments, the neutral heterogeneous catalyst can include one or more crystalline metal aluminosilicates. The crystalline metal aluminosilicates can be molecular sieves. The molecular sieves can be 5A molecular sieves.

In certain embodiments, the temperature of the disclosed processes can be in a range from about 20° C. to about 80° C. In certain embodiments, the temperature can be in a range from about 20° C. to about 25° C.

### BRIEF DESCRIPTION OF THE DRAWINGS

FIG. 1 is a GC-MS chromatogram showing formation of diacetone alcohol from acetone over time according to an exemplary process of the present disclosure.

FIG. 2 is a graphical representation of formation of diacetone alcohol from acetone over time according to an exemplary process of the present disclosure.

FIG. 3 is a GC-MS chromatogram showing formation of mesityl oxide from acetone over time according to an exemplary process of the present disclosure.

FIG. 4 is a graphical representation of formation of mesityl oxide from acetone over time according to an exemplary process of the present disclosure.

FIG. 5 is a graphical representation of formation of diacetone alcohol from acetone at different temperatures, according to exemplary processes of the present disclosure.

FIG. 6 is a graphical representation of formation of mesityl oxide from acetone at different temperatures, according to exemplary processes of the present disclosure.

FIG. 7 is a graphical representation of the ratio of diacetone alcohol to mesityl oxide formed from acetone at different temperatures, according to exemplary processes of the present disclosure.

FIG. 8 is a graphical representation of formation of diacetone alcohol from acetone catalyzed by different quantities of 5A molecular sieves, according to exemplary processes of the present disclosure.

FIG. 9 is a graphical representation of formation of mesityl oxide from acetone catalyzed by different quantities of 5A molecular sieves, according to exemplary processes of the present disclosure.

FIG. 10 is a graphical representation of the ratio of diacetone alcohol to mesityl oxide formed from acetone, as catalyzed by different quantities of 5A molecular sieves, according to exemplary processes of the present disclosure.

FIG. 11 is a GC-MS chromatogram showing formation of diacetone alcohol from acetone according to exemplary processes of the present disclosure.

FIG. 12 is a GC-MS chromatogram showing formation of diacetone alcohol and mesityl oxide from acetone according to an exemplary process of the present disclosure.

### DETAILED DESCRIPTION

The presently disclosed subject matter provides processes for synthesizing diacetone alcohol and/or mesityl oxide. An exemplary process includes contacting acetone with a heterogeneous catalyst at a temperature of less than 100° C. and can include contacting acetone with molecular sieves.

Acetone of various levels of purity can be used. In certain embodiments, acetone of less than or equal to 90% purity, greater than 90% purity, greater than 95% purity, greater than 97% purity, greater than 98% purity, greater than 99% purity, greater than 99.5% purity, or greater than 99.9% purity can be used. In certain embodiments, acetone of 99% purity or greater can be used.

The processes can be carried out in various reactors and reaction vessels known in the art. For example, the processes can be carried out in flasks, vials, reactors, including flow reactors, and/or tubes. The processes of the present disclosure can be carried out in batch and/or semi-continuous modes.

In certain embodiments, processes for synthesizing mesityl oxide can include contacting diacetone alcohol or a mixture of diacetone alcohol and acetone with a heterogeneous catalyst. Diacetone alcohol can be contacted with a heterogeneous catalyst at a temperature of less than 100° C., e.g., at a temperature in a range from about 20° C. to about 80°, or in a range from about 20° C. to about 25° C., to provide mesityl oxide.

Contacting acetone with a heterogeneous catalyst can include stirring, agitating, mixing, and/or flowing acetone in the presence of a heterogeneous catalyst. However, the processes of the present disclosure do not require stirring, agitating, flowing, or mixing; simply allowing acetone to stand in the presence of a heterogeneous catalyst can generate diacetone alcohol and mesityl oxide.

The heterogeneous catalysts can be various solid catalysts known in the art. One heterogeneous catalyst can be used, or a combination of heterogeneous catalysts can be used. By way of non-limiting example, suitable heterogeneous catalysts can include one or more metal salts, metalloid oxides, and/or metal oxides, e.g., titanium oxide, zirconium oxide, silica (silicon oxide), alumina (aluminum oxide), aluminosilicates, iron oxide, calcium oxide, calcium chloride, magnesium oxide, and magnesium chloride. Suitable heterogeneous catalysts can include one or more ceramics, glasses, and/or clays. In certain embodiments, the heterogeneous catalyst can be chosen for its high surface area. In certain embodiments, the heterogeneous catalyst can include one or more metal aluminosilicates. The metal aluminosilicates can be crystalline metal aluminosilicates or non-crystalline metal aluminosilicates. The metal aluminosilicates can be zeolites.

As used herein, the term “neutral” heterogeneous catalysts includes catalysts that are not strongly basic or acidic. Examples of heterogeneous catalysts that are strongly basic can include alkali hydroxides (e.g., sodium hydroxide, potassium hydroxide) and alkaline earth hydroxides (e.g., magnesium hydroxide or barium hydroxide). Neutral heterogeneous catalysts can have mild basic or acidic character but are not strongly basic or acidic. Certain heterogeneous catalysts can be rendered neutral by neutralization of strongly basic or strongly acidic activity; for example, alumina with strongly basic or acidic character can be treated with acid and/or base to neutralize any strongly basic or strongly acidic activity. Examples of neutral heterogeneous catalysts can include metal salts, metalloid oxides, and/or metal oxides, e.g., titanium oxide, zirconium oxide, silica (silicon oxide), alumina (aluminum oxide), aluminosilicates, iron oxide, magnesium sulfate, sodium sulfate, calcium oxide, calcium chloride, magnesium oxide, and magnesium chloride.

Crystalline metal aluminosilicates can be molecular sieves. Molecular sieves are solid materials with holes or openings of defined size that can adsorb small molecules of appropriate size. Molecular sieves can be used as desiccants, i.e., as materials useful for trapping and/or removing water. Molecular sieves can be neutral heterogeneous catalysts. Molecular sieves are frequently described in terms of the diameter of their pores. According to IUPAC notation, microporous molecular sieves can have pore diameters of less than 2 nm (20 Å), mesoporous molecular sieves can have pore diameters of between 2 nm and 50 nm (20 Å to 500 Å), and macroporous molecular sieves can have pore diameters of greater than 50 nm (500 Å).

Molecular sieves can be characterized by their pore diameter. For example, 3A molecular sieves have a pore diameter of about 3 Å (0.3 nm). 4A molecular sieves have a pore diameter of about 4 Å (0.4 nm). 5A molecular sieves have a pore diameter of about 5 Å (0.5 nm). 10X molecular sieves have a pore diameter of about 8 Å (0.8 nm). 13X molecular sieves have a pore diameter of about 10 Å (1.0 nm).

Molecular sieves can include numerous different types of materials. Molecular sieves can include aluminosilicates (zeolites). However, molecular sieves are not limited to aluminosilicates. Molecular sieves can be prepared from glasses, activated carbon (activated charcoal), clays (e.g., montmorillonites), aluminas, and/or silicas.

Certain molecular sieves can have basic or acidic character, but most molecular sieves can be considered to be neutral materials. In other words, molecular sieves can be neutral heterogeneous catalysts. In certain embodiments of the presently disclosed subject matter, molecular sieves can be treated with acid and/or base prior to use to neutralize any strongly basic or strongly acidic activity. In certain embodiments, molecular sieves that include aluminosilicates can have mild acidic character; such molecular sieves can be treated with base prior to use to neutralize acidic activity.

Molecular sieves can include an amount of adsorbed water. The amount of adsorbed water on molecular sieves can be in a range from about 18% to about 25%, by weight. Molecular sieves can be dehydrated by exposure to high temperature and/or reduced pressure. That is, adsorbed water can be removed from molecular sieves by exposure to high temperature and/or reduced pressure.

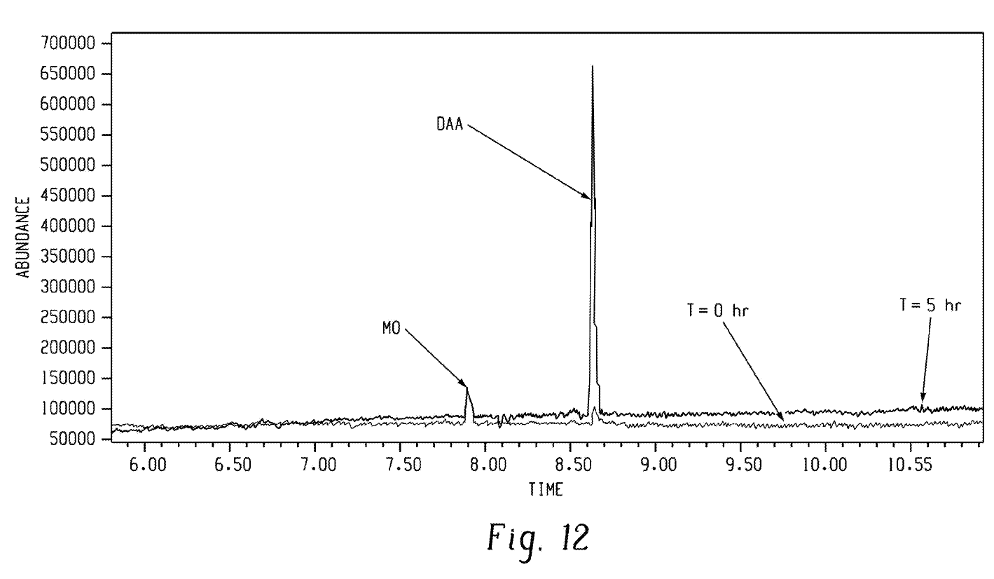

In certain embodiments, molecular sieves can be regenerated and reused after a reaction. For example, molecular sieves can be regenerated by heating to about 120° C., about 150° C., about 175° C., about 200° C., or above 200° C. while purging with a dry gas (e.g., dry air and/or nitrogen) or exposing the molecular sieves to reduced pressure (e.g., a pressure below about 100 torr, below about 25 torr, or below about 10 torr). After regeneration, molecular sieves can be cooled and stored under a dry atmosphere (e.g., dry air and/or nitrogen). By way of non-limiting example, the data of Example 5 and FIG. 12 suggest that 5A molecular sieves can be used to prepare diacetone alcohol and mesityl oxide from acetone, regenerated, and then reused in further reaction of acetone to prepare diacetone alcohol and mesityl oxide.

In certain embodiments, fresh molecular sieves can be dried prior to use to remove adsorbed water. Molecular sieves can be dried by heating to about 120° C., about 150° C., about 175° C., about 200° C., or above 200° C. while purging with a dry gas (e.g., dry air and/or nitrogen) or exposing the molecular sieves to reduced pressure (e.g., a pressure below about 100 torr, below about 25 torr, or below about 10 torr). After drying, molecular sieves can be cooled and stored under a dry atmosphere (e.g., dry air and/or nitrogen).

The molecular sieves used in the processes for synthesizing diacetone alcohol and mesityl oxide can be 3A, 4A, 5A, 10X, or 13X molecular sieves. In certain embodiment, the molecular sieves can be 5A molecular sieves. 5A molecular sieves can be abbreviated as “MS-5A.”

In accordance with the disclosed subject matter, 5A molecular sieves can catalyze the reaction of acetone to diacetone alcohol and mesityl oxide. The present disclosure includes the unexpected discovery that allowing acetone to stand at ambient temperature and pressure for about 5 hours can produce significant quantities of diacetone alcohol and mesityl oxide.

In certain embodiments, the temperature of the disclosed processes can be in a range from about 20° C. to about 100° C. For example, the temperature can be about 20° C., about 30° C., about 40° C., about 50° C., about 60° C., about 70° C., about 80° C., about 90° C., or about 100° C. In certain embodiments, the temperature of the disclosed processes can be in a range from about 20° C. to about 80° C. In certain embodiments, the temperature can be room temperature or ambient temperature, i.e., a temperature in a range from 20° C. to about 25° C.

In certain embodiments, the temperature of the process can be varied. For example, in certain embodiments, the process can be conducted at one temperature for an interval of time to optimize formation of diacetone alcohol, and the reaction temperature can then be changed to a second temperature for a second interval of time to optimize formation of mesityl oxide. By way of non-limiting example, the data of Example 2, Table 1, and FIG. 7 suggest that a process can include a first interval at a first, relatively low temperature (e.g., room temperature) to promote formation of diacetone alcohol and a second interval at a second, relatively high temperature (e.g., 60° C. or 80° C.) to promote formation of mesityl oxide.

In certain embodiments, the processes for synthesizing diacetone alcohol and mesityl oxide of the present disclosure can be conducted at ambient pressure, i.e., at a pressure of about 760 torr (1 atm).

After synthesis of diacetone alcohol and/or mesityl oxide, the heterogeneous catalyst can be removed from the reaction mixture by various methods known in the art, e.g., filtration and/or centrifugation.

In certain embodiments of the present disclosure, processes for synthesizing diacetone alcohol and/or mesityl oxide can include contacting acetone with one or more desiccants, e.g., calcium chloride, magnesium sulfate, calcium sulfate, or sodium sulfate.

Diacetone alcohol, mesityl oxide, and acetone can be separated from one another by various methods known in the art. In certain embodiments, diacetone alcohol (boiling point=166° C.), mesityl oxide (boiling point =129.5° C.), and acetone (boiling point=56° C.) can be separated from one another by fractional distillation.

In certain embodiments, processes for synthesizing diacetone alcohol and/or mesityl oxide can be conducted for less than 10 minutes, about 10 minutes to about 1 hour, about 1 hour to about 2 hours, about 2 hours to about 4 hours, about 4 hours to about 8 hours, about 8 hours to about 16 hours, about 16 hours to about 1 day, about 1 day to about 2 days, about 2 days to about 4 days, about 4 days to about 1 week, or longer than 1 week. The reaction time can be about 5 hours. Reaction time can be dependent on reaction temperature.

### EXAMPLES

The following non-limiting examples are included purely by way of illustration of the presently disclosed subject matter. The acetone used in the following examples was of 99% purity.

### Example 1

### Time Studies of the Synthesis of Diacetone Alcohol and Mesityl Oxide From Acetone

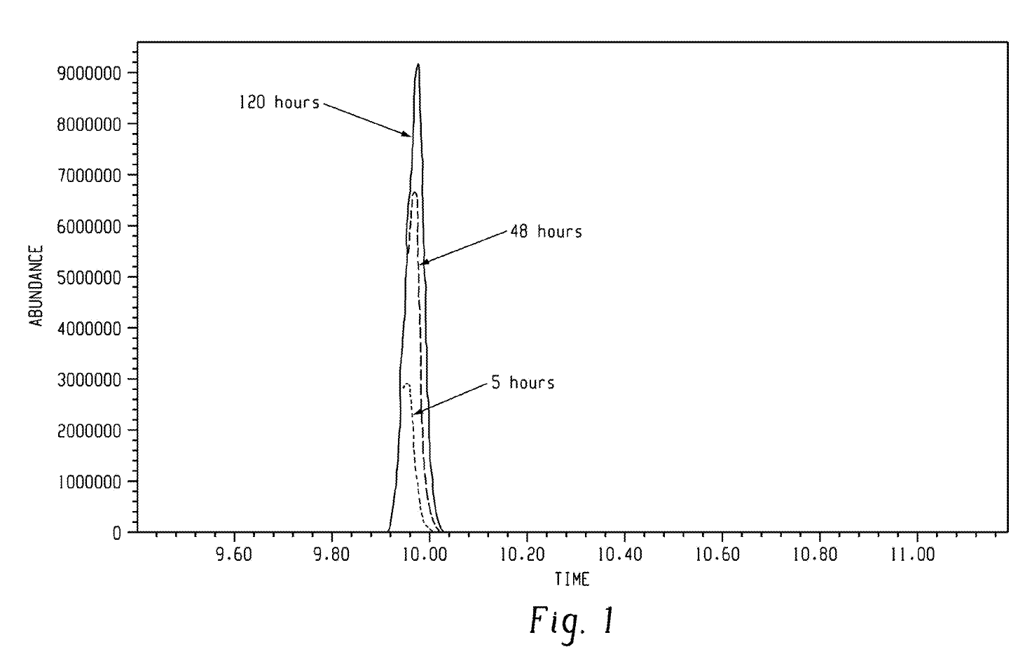

Approximately 15 g of acetone was added to a bed (10 g) of 5A molecular sieves (MS-5A) in a 40 mL screw-cap vial. The vial was placed on a rotator and rotated for about 5 hours at room temperature and allowed to stand for the rest of the study time (5 days). Approximately 1.5 mL of the reaction mixture was withdrawn at 5, 48, and 120 hours and filtered for GCMS analysis. The results of these experiments are shown in FIGS. 1-4.

FIG. 1 is a GC-MS total ion chromatogram of diacetone alcohol. FIG. 1 shows an increase of diacetone alcohol concentration with time, from 5 hours to 48 hours to 120 hours.

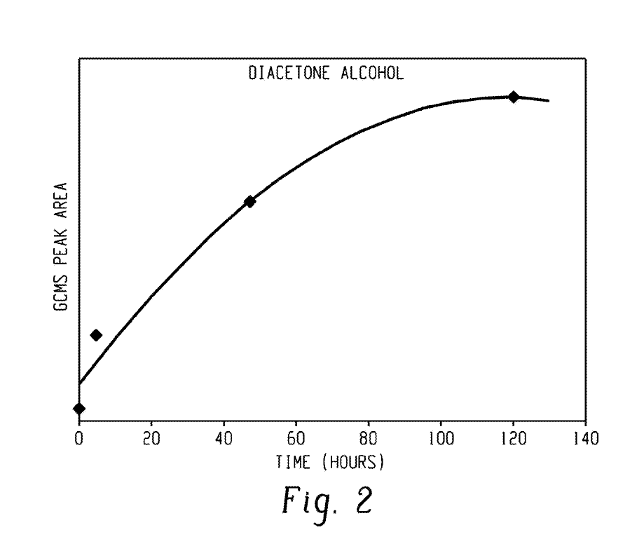

FIG. 2 is a graphical representation of the data presented in FIG. 1, showing the rise in concentration of diacetone alcohol over time. The curve shown in FIG. 2 was fitted to the data using the Polynomial (Order 2) curve-fitting feature of Microsoft Excel.

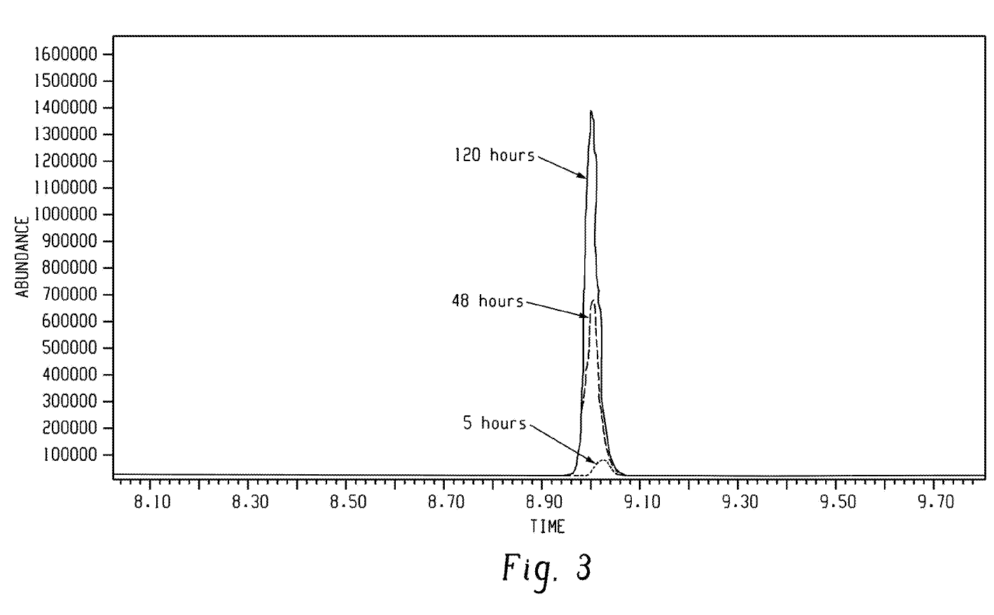

FIG. 3 presents a GC-MS total ion chromatogram of mesityl oxide. FIG. 3 shows an increase of mesityl oxide concentration with time, from 5 hours to 48 hours to 120 hours.

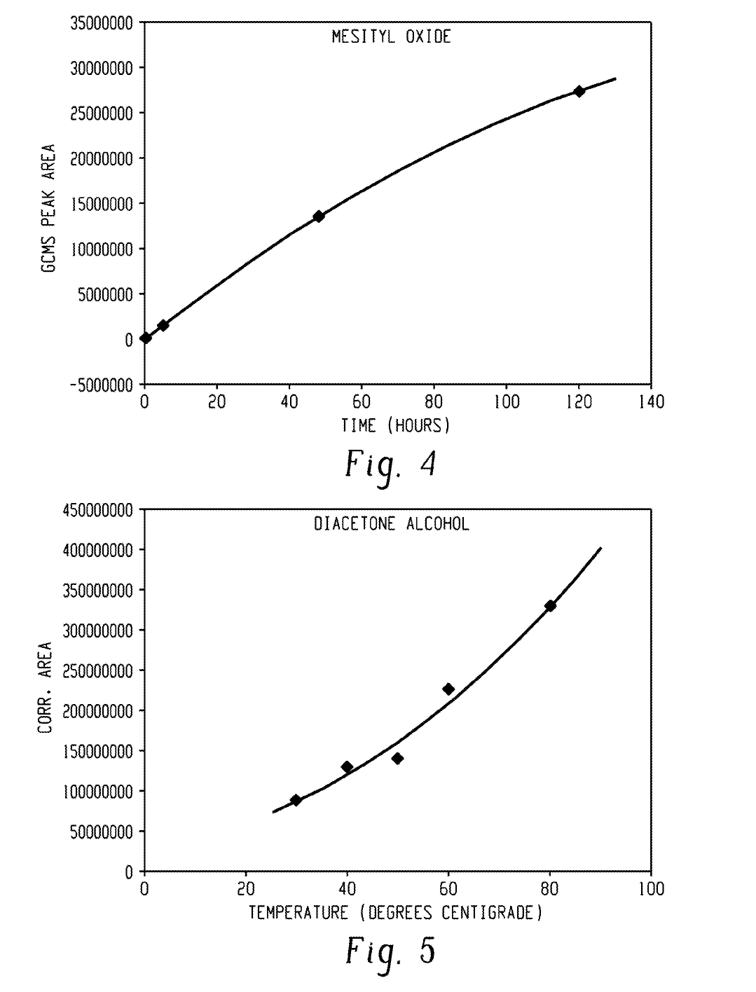

FIG. 4 is a graphical representation of the data presented in FIG. 3, showing the rise in concentration of mesityl oxide over time. The curve shown in FIG. 4 was fitted to the data using the Polynomial (Order 2) curve-fitting feature of Microsoft Excel.

FIGS. 2 and 4 indicate that while concentration of diacetone alcohol under the reaction conditions described above can reach a peak at about 120 hours, the concentration of mesityl oxide can continue to rise. Mesityl oxide can be formed from diacetone alcohol. Without being bound to any particular theory, it can be that at around 120 hours, under the reaction conditions described above, the rate of formation of diacetone alcohol from acetone is approximately equal to the rate of formation of mesityl oxide from diacetone alcohol.

### Example 2

### Temperature Studies of the Synthesis of Diacetone Alcohol and Mesityl Oxide From Acetone

Temperature studies were carried out at 30° C., 40° C., 50° C., 60° C., and 80° C. In these experiments, approximately 15 g of acetone was added to 10 g of MS-5A and placed in an oven at the given temperature. The reactions were allowed to run for 5 hours without any stirring or agitation. Approximately 1.5 mL of the sample was then filtered through a 0.45 micron PTFE filter and analyzed by GC-MS and GC-FID.

FIG. 5 is a graphical representation showing the rise in concentration of diacetone alcohol with increasing temperature, as determined by GC-MS and GC-FID.

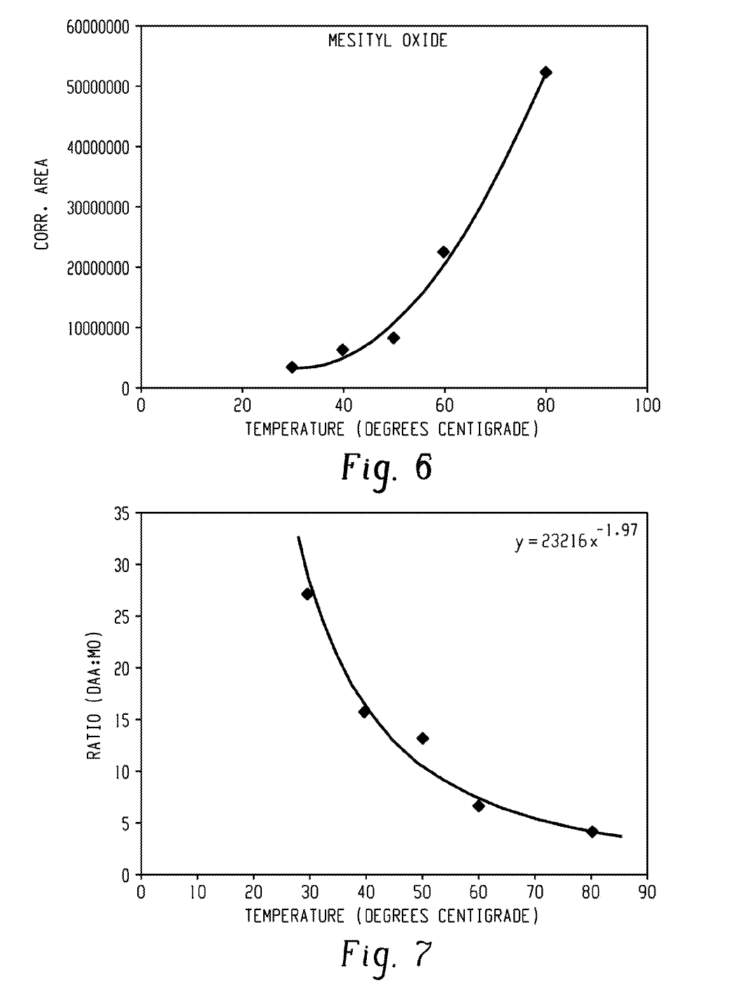

FIG. 6 is a graphical representation showing the rise in concentration of mesityl oxide with increasing temperature, as determined by GC-MS and GC-FID.

Table 1 presents the ratio of the concentration of diacetone alcohol to the concentration of mesityl oxide (DAA:MO, mole:mole) as a function of temperature, according to the experiments described above.

TABLE 1

| Temperature (° C.) | Ratio DAA:MO (mole:mole) |
|---|---|
| 30 | 27.1 |
| 40 | 15.7 |
| 50 | 13 |
| 60 | 6.6 |
| 80 | 4 |

FIG. 7 is a graphical representation of the data presented in Table 1, showing the decline in the ratio of the concentration of diacetone alcohol to the concentration of mesityl oxide (DAA:MO, mole:mole) as reaction temperature increases.

It has been reported that synthetic yields of diacetone alcohol tend to be higher at lower temperature. See G. G. Podrebarac, F. T. T. Ng, and G. L. Rempel, Chem. Eng. Sci., 1998, 53, 1067. Surprisingly, the data of Example 2 and FIGS. 5 and 6 indicates that yields of both diacetone alcohol and mesityl oxide can increase with increasing temperature.

Example 3

### Studies on the Quantity of 5A Molecular Sieves in the Synthesis of Diacetone Alcohol and Mesityl Oxide From Acetone

Twenty (20) grams of acetone was added to each of four samples containing, respectively, 5, 10, 15, and 20 grams of MS-5A. These were placed in an oven at 60° C. for 5 hours without stirring or other agitation. 1.5 mL of each sample was then filtered and analyzed by GC-MS and GC-FID.

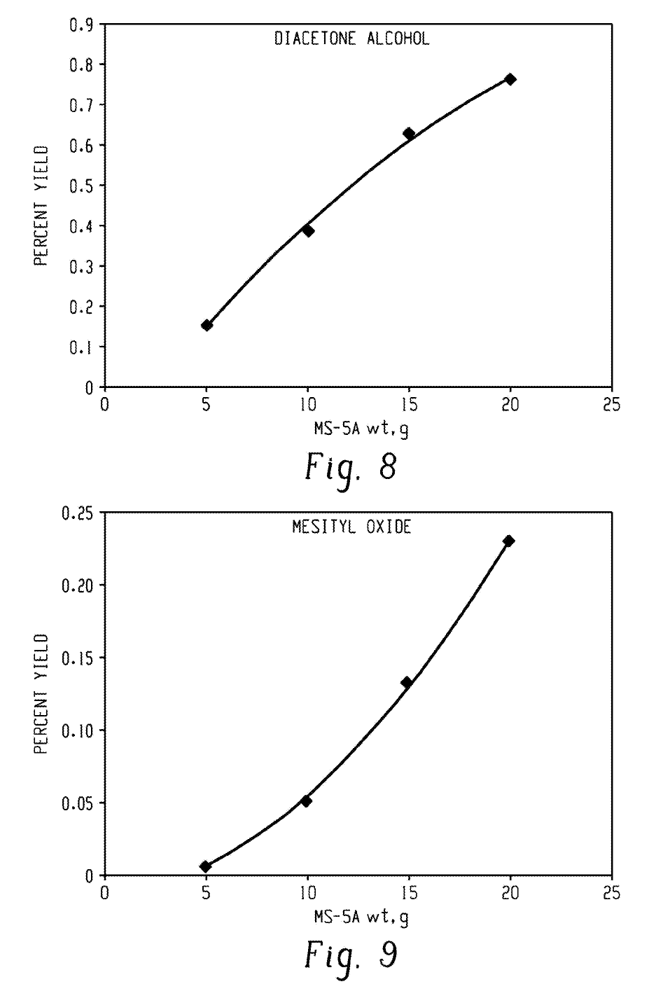

FIG. 8 is a graphical representation showing the rise in concentration of diacetone alcohol with increasing quantities of MS-5A, as determined by GC-MS and GC-FID. FIG. 8 indicates that yields of diacetone alcohol of up to about 0.8% can be obtained.

FIG. 9 is a graphical representation showing the rise in concentration of mesityl oxide with increasing quantities of MS-5A, as determined by GC-MS and GC-FID. FIG. 9 indicates that yields of diacetone alcohol of up to about 0.25% can be obtained.

Table 2 presents the ratio of the concentration of diacetone alcohol to the concentration of mesityl oxide (DAA:MO, mole:mole) as a function of the quantity of MS-5A, according to the experiments described above.

TABLE 2

| MS-5A weight (g) | Ratio DAA:MO (mole:mole) |
|---|---|
| 5 | 20.9 |
| 10 | 7.6 |
| 15 | 4.8 |
| 20 | 3.3 |

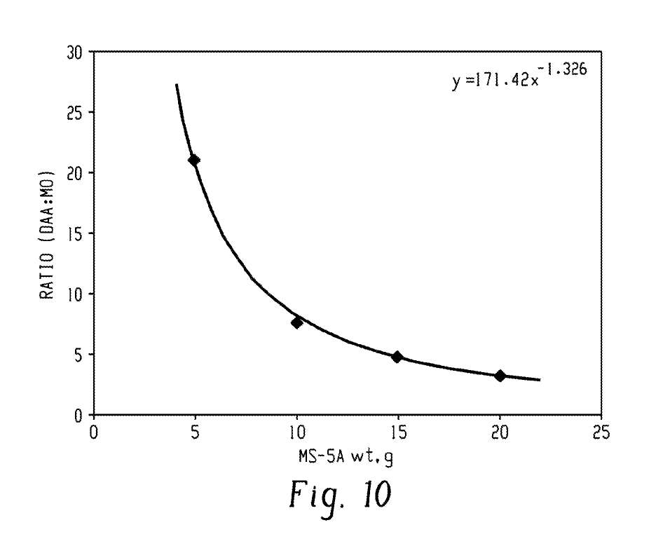

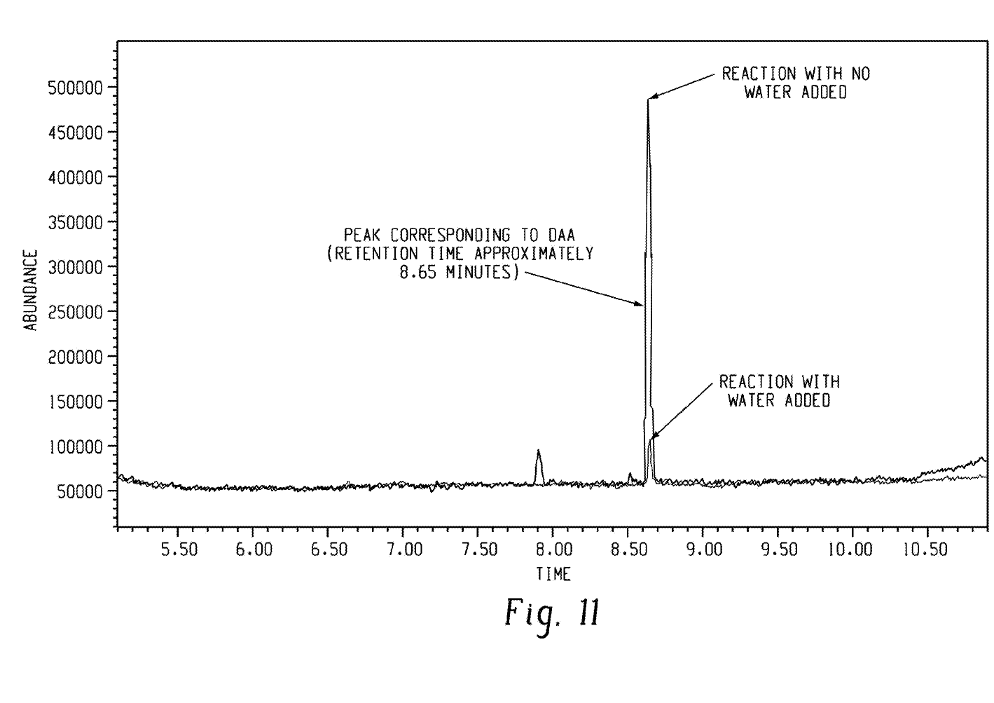

FIG. 10 is a graphical representation of the data presented in Table 2, showing the decline in the ratio of the concentration of diacetone alcohol to the concentration of mesityl oxide (DAA:MO, mole:mole) as the quantity of MS-5A increases.

The data of Example 3 and FIGS. 8 and 9 indicate that yields of diacetone alcohol and mesityl oxide can increase with increasing quantities of MS-5A.

The data of Examples 2 and 3, Tables 1 and 2, and FIGS. 7 and 10 indicate that the processes of the present disclosure allow for optimization of the ratio of diacetone alcohol to mesityl oxide. The temperature of the reaction and/or the quantity of MS-5A can be modified to optimize reaction selectivity for one product over the other, if so desired.

### Example 4

### Study on Drying of 5A Molecular Sieves and Addition of Water in the Synthesis of Diacetone Alcohol and Mesityl Oxide From Acetone

A quantity of MS-5A was dried overnight at 180° C. to provide dried MS-5A. A sample of 10 g of dried MS-5A was then prepared, to which was added 1 g of water, and the resulting mixture was warmed to 60° C. and held at 60° C. overnight. A 10 g sample of acetone was then added, and the resulting mixture was held at 60° C. for 5 hours. The composition of the reaction mixture (“Reaction with Water Added”) was then determined by GC-MS.

A second 10 g sample of dried MS-5A was also prepared, to which was added 10 g of acetone. Water was not added to this mixture. The mixture was heated to 60° C. for 5 hours, and the composition of the reaction mixture (“Reaction with No Water Added”) was then determined by GC-MS.

FIG. 11 presents the GC-MS results of these experiments. More DAA was formed in the Reaction with No Water Added than in the Reaction with Water Added, which indicates that addition of water can reduce the activity of the MS-5A.

### Example 5

### Study on Regeneration of 5A Molecular Sieves

A sample of MS-5A was contacted with acetone to prepare diacetone alcohol and mesityl oxide. The MS-5A were then removed from the reaction mixture and regenerated by heating to 150° C. for 3 hours, to provide regenerated MS-5A. Acetone was then contacted with the regenerated MS-5A at 60° C. for 5 hours, to provide diacetone alcohol and mesityl oxide. FIG. 12 presents the GC-MS results of this experiment. The GC-MS chromatogram indicates that diacetone alcohol (DAA) and mesityl oxide (MO) were formed.

The processes disclosed herein can have numerous advantages over certain existing technologies, including greater selectivity and control over the ratio of diacetone alcohol to mesityl oxide. In some embodiments, the processed have one or more of greater efficiency, cheaper catalysts, reduced input of energy, and milder conditions. For example, in certain embodiments, use of a neutral heterogeneous catalyst can reduce the quantities of side products formed during reaction of acetone. Certain existing reactions of acetone can cause formation of triacetone dialcohol. See, for example, U.S. Pat. No. 5,672,764, which describes formation of triacetone dialcohol during preparation of diacetone alcohol from acetone. An advantage of the use of a neutral heterogeneous catalyst for preparation of diacetone alcohol and/or mesityl oxide can be the absence of formation of triacetone dialcohol. Triacetone dialcohol was not observed as a product of reaction of acetone in the presence of 5A molecular sieves at 30° C., 40° C., 50° C., 60° C., and 80° C.

The processes are further illustrated by the following Embodiments.

### Embodiment 1

A process for synthesizing diacetone alcohol, comprising contacting acetone with a neutral heterogeneous catalyst at a temperature of less than 100° C.

### Embodiment 2

The process of Embodiment 1, wherein the neutral heterogeneous catalyst comprises one or more crystalline metal aluminosilicates.

### Embodiment 3

The process of Embodiment 2, wherein the crystalline metal aluminosilicates are molecular sieves.

### Embodiment 4

The process of Embodiment 1 or Embodiment 2 or Embodiment 3, wherein the temperature is in a range from 20° C. to 80° C., preferably in a range from 20° C. to 25° C.

### Embodiment 5

A process for synthesizing diacetone alcohol, comprising contacting acetone with molecular sieves.

### Embodiment 6

The process of Embodiment 5, wherein the molecular sieves are 5A molecular sieves.

### Embodiment 7

A process for synthesizing mesityl oxide, comprising contacting acetone with a neutral heterogeneous catalyst at a temperature of less than 100° C.

### Embodiment 8

The process of Embodiment 8, wherein the neutral heterogeneous catalyst comprises one or more crystalline metal aluminosilicates.

### Embodiment 9

The process of Embodiment 8, wherein the crystalline metal aluminosilicates are molecular sieves.

### Embodiment 10

The process of Embodiment 7, or Embodiment 8, or Embodiment 9, wherein the temperature is a range from 20° C. to 80° C., preferably wherein the temperature is in a range from 20° C. to 25° C.

### Embodiment 11

A process for synthesizing mesityl oxide, comprising contacting acetone with molecular sieves.

### Embodiment 12

The process of Embodiment 11, wherein the molecular sieves are 5A molecular sieves.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

Although the presently disclosed subject matter and its advantages have been described in detail, it should be understood that various changes, substitutions, and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

What is claimed is:

1. A process for synthesizing diacetone alcohol, comprising contacting acetone with a neutral heterogeneous catalyst at a temperature of less than 100° C.

2. The process of claim 1, wherein the neutral heterogeneous catalyst comprises one or more crystalline metal aluminosilicates.

3. The process of claim 2, wherein the crystalline metal aluminosilicates are molecular sieves.

4. The process of claim 1, wherein the temperature is in a range from about 20° C. to about 80° C., preferably in a range from about 20° C. to about 25° C.

5. A process for synthesizing diacetone alcohol, comprising contacting acetone with molecular sieves.

6. The process of claim 5, wherein the molecular sieves are 5A molecular sieves.

7. A process for synthesizing mesityl oxide, comprising contacting acetone with a neutral heterogeneous catalyst at a temperature of less than 100° C.

8. The process of claim 7, wherein the neutral heterogeneous catalyst comprises one or more crystalline metal aluminosilicates.

9. The process of claim 8, wherein the crystalline metal aluminosilicates are molecular sieves.

10. The process of claim 7, wherein the temperature is a range from about 20° C. to about 80° C., preferably wherein the temperature is in a range from about 20° C. to about 25° C.

11. A process for synthesizing mesityl oxide, comprising contacting acetone with molecular sieves.

12. The process of claim 11, wherein the molecular sieves are 5A molecular sieves.

13. The process of claim 4, wherein the temperature is in a range from from about 20° C. to about 25° C.

14. The process of claim 10, wherein the temperature is a range from about 20° C. to about 25° C.
